(19) United States
(12) Patent Application Publication
(10) Pub. No.: US 2021/0098643 A1
(43) Pub. Date: Apr. 1, 2021

(54) LIGHT SENSITIVE ELEMENT, IMAGING ELEMENT, AND IMAGING DEVICE

(71) Applicants: NATIONAL INSTITUTE OF ADVANCED INDUSTRIAL SCIENCE AND TECHNOLOGY, Tokyo (JP); NIKON CORPORATION, Tokyo (JP)

(72) Inventors: Yuzuru IKEHARA, Tsukuba-shi (JP); Mutsuo OGURA, Tsukuba-shi (JP); Sanae IKEHARA, Tsukuba-shi (JP); Kenji MIYAMOTO, Yokohama-shi (JP); Yutaka IWASAKI, Yokohama-shi (JP)

(73) Assignees: NATIONAL INSTITUTE OF ADVANCED INDUSTRIAL SCIENCE AND TECHNOLOGY, Tokyo (JP); NIKON CORPORATION, Tokyo (JP)

(21) Appl. No.: 17/119,117

(22) Filed: Dec. 11, 2020

Related U.S. Application Data
(63) Continuation of application No. PCT/JP2019/022169, filed on Jun. 4, 2019.

(30) Foreign Application Priority Data
Jun. 14, 2018 (JP) 2018-113547

(51) Int. Cl. H01L 31/109; H01L 31/0352; H01L 31/12

(52) U.S. Cl. CPC H01L 31/109; H01L 31/12; H01L 31/035209

(57) ABSTRACT

This light sensitive element comprises: a substrate; a light absorbing layer containing InGaA, the light absorbing layer being disposed on the substrates; and semiconductor layers containing InAsP. The semiconductor layers are disposed on an upper surface and on a lower surface of the light absorbing layer, respectively. The semiconductor layers constitutes a quantum well structure with the light absorbing layer.

### TECHNICAL FIELD

The present disclosure relates to a light sensitive element, an imaging element, and an imaging device.

### BACKGROUND ART

There has been developed an imaging element including a plurality of light sensitive elements that detects a light in an infrared wavelength region and outputs it as an image (for example, see Patent Literature 1). Generally, the infrared-compatible imaging element includes a Si crystal type and an InGaAs crystal type, and the imaging element using the InGaAs crystal has sensitivity to a light in a longer wavelength area than that of the Si crystal type. As one example, while the upper limit of the sensitivity of the Si crystal type is around. 1000 nm, the InGaAs crystal type has sensitivity up to around 1700 nm. The InGaAs crystal type imaging element has been desired to have a sufficient high sensitivity in a wide wavelength region in an infrared region.

### CITATION LIST

### Patent Literature

Patent Literature 1: JP 2002-373999 A

### BRIEF DESCRIPTION OF DRAWINGS

FIG. 1 is a cross-sectional view illustrating an outline of a configuration of a light receiving element 2000 of a first embodiment.

FIG. 2 is a table showing results of measuring sensitivity (wavelength region in which the sensitivity of 80% or more is obtained) of respective three kinds of elements A, B, and C having different composition ratios of In to Ga in InGaAs of a fourth layer 204 in the light receiving element 2000 of the embodiment.

FIG. 3A is a graph illustrating changes in wavelength and relative sensitivity of the elements A, B, and C according to the embodiment.

FIG. 3B illustrates the relative sensitivities at a wavelength of 1500 nm of the elements A, B, and C according to the embodiment.

FIG. 3C is a table showing various kinds of properties of the elements A, B, and C according to the embodiment.

FIG. 4 is a graph illustrating a relation between wavelength and absorption coefficient in a lipid according to the embodiment.

FIG. 5 is a graph illustrating irradiance of illuminating light sources to obtain a required image contrast of the embodiment together with an upper limit value in a safety standard.

FIG. 6 is a drawing to describe an exemplary configuration of an imaging system 1 according to the first embodiment.

FIG. 7 is a schematic diagram of an optical system employable in an imager 30 according to the embodiment.

FIG. 8 is a schematic diagram describing an exemplary configuration of an endoscope system according to a second embodiment.

FIG. 9 is an enlarged view of the endoscope 600 of FIG. 8.

FIG. 10 in an external view of an imaging device 700 according to a third embodiment.

FIG. 11 is a drawing illustrating an internal configuration of the imaging device 700 of FIG. 10.

### DESCRIPTION OF EMBODIMENTS

The following describes embodiments with reference to the accompanying drawings. The accompanying drawings represent functionally same elements by same reference numerals in some cases. Although the accompanying drawings illustrate the embodiments and implementation examples according to a principle of the present disclosure, these drawings are for understanding of the present disclosure and never used for limited interpretation of the present disclosure. The descriptions of this specification are merely typical examples and therefore do not limit the claims or application examples of the present disclosure by any means.

While the embodiments give the description in detail enough for a person skilled in the art to carry out the present disclosure, it is necessary to understand that other implementations and configurations are possible and that changes in configurations and structures and substitutions of various components can be made without departing from the scope or spirit of the technical idea of the present disclosure. Therefore, the following description should not be interpreted to be limited.

### First Embodiment

Next, a light receiving element, an imaging element, and an imaging device according to the first embodiment will be described with reference to the drawings.

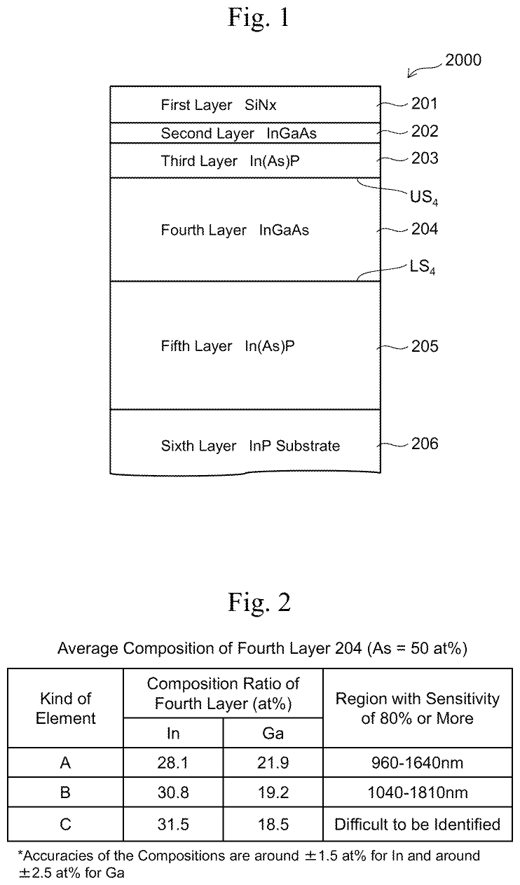

First, a light receiving element 2000 (example: the infrared light (near-infrared)-compatible light receiving element 2000) using a lattice mismatch type InGaAs crystal according to the first embodiment will be described with reference to FIG. 1 to FIG. 5.

FIG. 1 is a cross-sectional view illustrating an outline of a configuration of the light receiving element 2000 of the first embodiment. This light receiving element 2000 can be roughly configured of a first layer 201, a second layer 202, a third layer 203, a fourth layer 204, a fifth layer 205, and a sixth layer 206 (substrate) from the upper layer. Additionally, as illustrated in FIG. 1, the light receiving element 2000 has a multilayer structure in which a plurality of layers are laminated in a lamination direction (example: a thickness direction, single direction), and the multilayer structure is formed in the order of the first layer 201, the second layer 202, the third layer 203, the fourth layer 204, the fifth layer 205, and the sixth layer 206 (substrate) from the upper layer. Note that, in the following description, among surfaces in the lamination direction of the respective layers (surfaces perpendicular to the lamination direction), a surface facing the substrate is referred to as a “lower surface” and a surface that is on the opposite side of the lower surface and far when viewed from the substrate is referred to as an “upper surface.”

For example, a plurality of the light receiving elements 2000 arrayed in a matrix pattern or an array pattern on one substrate (or on a plurality of the substrates) form an imaging element (example: light receiving sensor), and the imaging element is embedded into a camera, and thus an imaging device or an imaging system can be configured.

The sixth layer 206 is a substrate made of, for example, indium phosphide (InP). The fifth layer 205 is a III-V group semiconductor layer made of, for example, indium arsenide phosphide (InAsP) as the main component. A thickness (lamination direction) of the fifth layer 205 can be set to, for example, around 2000 nm.

The fourth layer 204 is a light absorbing layer and a III-V group semiconductor layer made of indium gallium arsenide (inGaAs). For example, when the light receiving element 2000 is viewed from the side surface with the sixth layer 206 disposed on the lower side in the gravity direction, the fourth layer 204 as the light absorbing layer constitutes a quantum well structure together with the fifth layer 205, which is disposed on a lower surface LS₄ (example: a surface facing the sixth layer 206) of the fourth layer 204, and the third layer 203, which is disposed on an upper surface US₄. (example: a surface facing the first layer 201, a surface facing the second layer 202) of the fourth layer 204. The thickness of the fourth layer 204 can be set to be, for example, around 1500 nm. As described later, the fourth layer 204 includes a lattice mismatch type InGaAs crystal layer that contains indium gallium arsenide (InGaAs) as the main component, which is different from the fifth layer 205 (and/or the third layer 203). When a composition ratio of arsenide (As) in a composition of InGaAs is 50 at %, a composition ratio (at %, average) of Indium (In) to Gallium (Ga) in the InGaAs is set such that the former becomes higher than the latter, for example, In:Ga=30.8:19.2. Here, an accuracy of the composition of InGaAs by analysis is about ±1.5 at % for in and about ±2.5 at % for Ga.

Thus, an amount of Indium (In) is significantly larger than that of Gallium (Ga), and the fourth layer 204 has a lattice mismatch crystal structure. This composition ratio allows the light receiving element 2000 according to the embodiment to have sensitivity (light receiving sensitivity) up to a wavelength region exceeding 1800 nm in a wavelength region of infrared light (near-infrared light). A match of lattice constants between the respective layers to be laminated in epitaxial growth is referred to as a lattice match. To grow a crystal layer on the substrate, when a lattice constant of the substrate does not match a lattice constant of the crystal to be grown, the laminated crystal layer strains. In the embodiment, a layer where the lattice constant is unmatched is purposely disposed to strain the fourth layer 204 as the light absorbing layer. This strain allows obtaining the light receiving element having sufficient high sensitivity in the wide wavelength region of the infrared region.

As described in detail later, the inventors calculated, based on theoretical calculation, a value of the composition ratio of In to Ga where the sensitivity is obtained up to the wavelength region exceeding 1800 nm. Specifically, it was found from the result of the theoretical calculation that an energy having a light at 1750 nm is 0.709 eV. It was proved that the composition ratio of In to convert the light into electrons in the InGaAs element by photoelectric conversion needs to be at least 30 at % or more when the composition ratio of As in the InGaAs element is 50 at %. With such the composition ratio of In, an energy gap becomes smaller than 0.709 eV (example: around 0.6633 eV), and an electric signal can be obtained with the light at 1750 nm. Meanwhile, excessively increasing the composition ratio of In causes deterioration of stability of the InGaAs element. Further, even when a light does not enter the element, since free electrons are excited by, for example, lattice vibration, the imaging element produces a lot of noise, and therefore using it as the imaging element is possibly difficult.

In accordance with the result of the theoretical calculation as described above, the inventors actually prototyped elements having various In:Ga composition ratios in the fourth layers 204 and verified the properties. As a result, the followings have been found. In order to have a composition ratio of In to Ga that provides a high sensitivity in the wavelength region exceeding 1800 nm, and also provides a high stability, the composition ratio of In is 30 at % or more in terms of theoretical calculation as described above. In addition, as a result of the prototypes and the results of verification in accordance with this theoretical calculation value, the composition ratio (at %) of In to Ga is preferably 30.8:19.2.

Note that while the composition ratio (at %) of In to Ga of 30.8:19.2 in the fourth layer 204 may be the average value in the lamination direction of the fourth layer 204, the composition ratio is preferably uniform in the lamination direction. For example, excluding an interface with the third layer 203 and the fifth layer 205, the composition ratio is preferably uniform in the lamination direction in the ranges of the accuracies of the compositions described above.

Similarly to the fifth layer 205 and different from the fourth layer 204, the third layer 203 is, for example, a III-V group semiconductor layer made of indium arsenide phosphide (InAsP) as the main component. A thickness of the third layer 203 can be set to be smaller than a thickness of the fourth layer 204, for example, around 200 nm. Note that when the composition ratio of In to Ga in the InGaAs as the main component of the fourth layer 204 is set to be the composition ratio (at %) of 30.8:19.2 as described above, in the InAsP in the third layer 203, which is formed on the upper surface of the fourth layer 204 as the light absorbing layer, the composition ratio of In is 50 at % and the composition ratio (average, at %) of P to As in the remaining 50 at % can be about 42:8. Thus, interposing the InGaAs layer of the fourth layer 204 between the InAsP layers of the third layer 203 and the fifth layer 205 can form a structure that strains the InGaAs layer of the fourth layer 204 and confines generated electric charges.

For example, similarly to the fourth layer 204, the second layer 202 is made of indium gallium arsenide (InGaAs) as the main component. The thickness of the second layer 202 can be set to, for example, around 100 nm. The second layer 202 made of the InGaAs layer as the main component similarly to the fourth layer 204 allows adjusting a degree of strain generated in the fourth layer 204 and improving the sensitivity of the light receiving element.

The first layer 201 is a passivation film and can be made of silicon nitride (SiNx) as one example. The thickness of the first layer 201 can be set to, for example, around 200 nm. The first layer 201 has a role of protecting the second layer 202, which is the layer under the first layer 201, and the like.

The second layer 202 to the fifth layer 205 can be deposited by, for example, metal-organic vapor phase epitaxy method (MOVPE) or molecular beam epitaxy. The layers from the second layer 202 to the fifth layer 205 are configured of a plurality of (two in this case) InGaAs/InAsP layers (a set of layers of the InGaAs layers and the InAsP layers (combination layers, repeating layers)). The layer configuration of the light receiving element 2000 includes the repeating layers in which at least the two InGaAs/InAsP layers (example: a set of layers of the InGaAs layers and the InAsP layers formed on the surface (one of the opposite surfaces, or on either surface) of the substrate, in the order of the InAsP layer and the InGaAs layer from the substrate) are intermittently (discontinuously) or continuously formed. For example, in the light receiving element 2000, at least the two layers including the InGaAs layer (light absorbing layer) and the InAsP layer (semiconductor layer) are repeatedly formed. Additionally, the light receiving element 2000 may have the layer configuration in which the light absorbing layers and the semiconductor layers are each repeatedly formed across a plurality of layers. For example, the light receiving element 2000 has a structure in which the layers of different main components (example: the light absorbing layers, the semiconductor layers, and the like) are repeatedly stacked in alternation in the film thickness direction of the sixth layer 206 (substrate) (or, for example, the layer any of the second layer 202 to the fifth layer 205). Note that each of the third layer 203 to the fifth layer 205 are not limited to be one layer, and each of the layers can be repeatedly deposited across the plurality of layers.

Next, the composition and the composition ratio of the fourth layer 204 will be described with reference to FIG. 2 to FIG. 6.

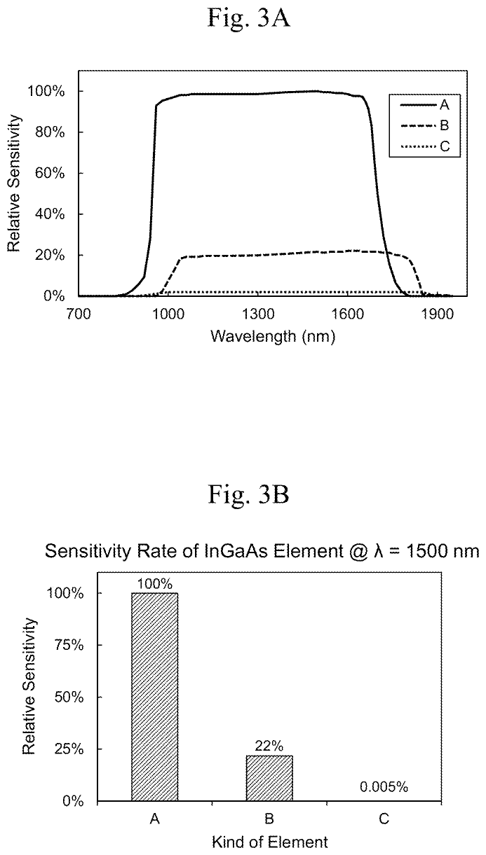

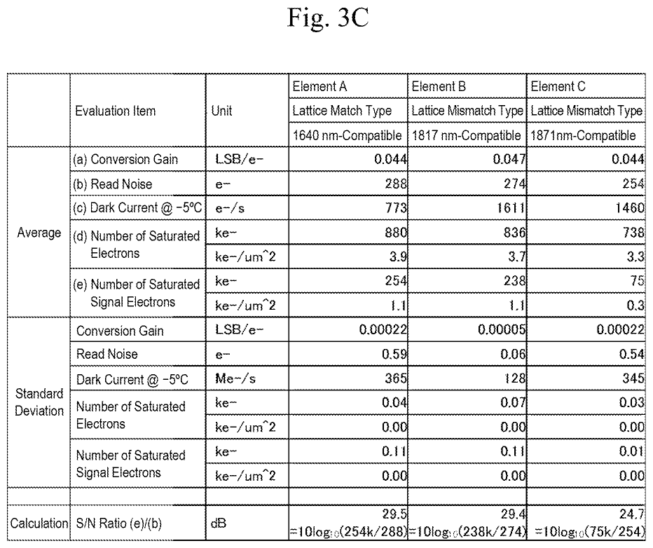

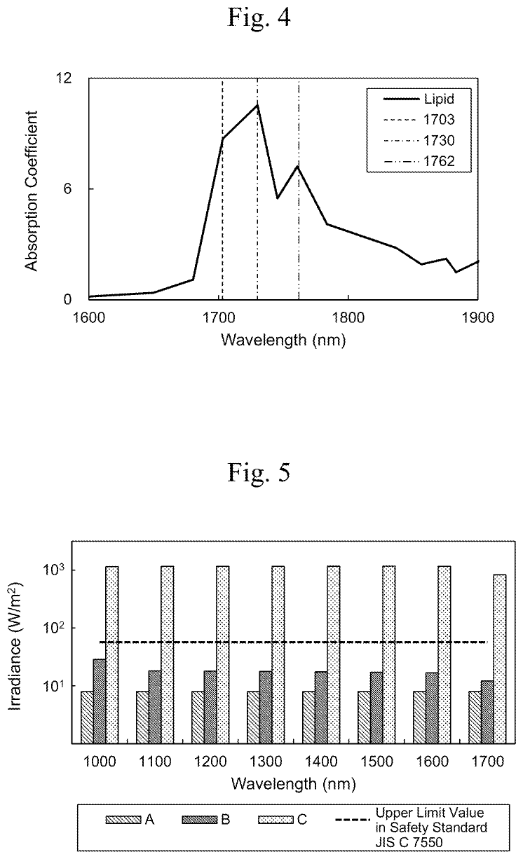

FIG. 2 is a table showing results of measuring sensitivity (wavelength region in which the sensitivity of 80% or more is obtained in this case) of received light of respective three kinds of elements A, B, and C having different composition ratios of in to Ga of the fourth layer 204 (light receiving elements in which configurations of the other layers are mostly the same). FIG. 3A is a graph illustrating changes in wavelength and relative sensitivity of the elements A, B, and C. FIG. 3B illustrates the relative sensitivities at a wavelength of 1500 nm of the elements A, B, and C. FIG. 3C is a table showing various kinds of properties of the elements A, B, and C. Note that the relative sensitivity here means the relative sensitivity when the maximum value of the sensitivity of the element A is set as the reference (100%). FIG. 4 is a graph illustrating a relation between wavelength and absorption coefficient in a lipid. FIG. 5 is a graph illustrating irradiance of illuminating light sources to obtain a required image contrast together with an upper limit value in a safety standard.

All of the elements A, B, and C have the composition ratio of arsenide (As) of 50 at % in the composition of InGaAs, and the composition ratio (average) of In to Ga in the remaining 50 at % is as shown in FIG. 2 (28.1:21.9, 30.8:19.2, 31.5:18.5). The element A is an element having a design value of 26.5:23.5 (at %) and a measurement value not meeting the above-described range of theoretical calculation (the composition ratio of In is 30 at % or more). The element B and the element C are elements prototyped so as to meet the above-described range of theoretical calculation. It is assumed that In has an error of around ±1.5 at % and Ga has an error of around ±2.5 at %. Note that the composition ratios of the respective elements A, B, and C were measured (analyzed) using the Rutherford backscattering spectrometry method (RBS).

It was found that, when the composition ratio of arsenide (As) in the composition of InGaAs was 50 at %, the composition ratio (average) of In to Ga was 28.1:21.9 in the element A, and, as a result of measurement, the sensitivity of 80% or more in a comparison with sensitivity at the peak was obtained in the wavelength region from 960 nm to 1640 nm (see FIG. 2 and FIG. 3A). In this case, the sensitivity at the peak means the maximum sensitivity obtained in the light receiving element in which the element A is used in the fourth layer 204.

Meanwhile, it was found that, when the composition ratio of arsenide (As) in the composition of InGaAs was 50 at %, the composition ratio (average) of In to Ga was 30.8:19.2 in the element B, and, as a result of measurement, the sensitivity of 80% or more in a comparison with the sensitivity at the peak (also referred to as the peak of the sensitivity) was obtained in the wavelength region from 1040 nm to 1810 nm. In this case, the sensitivity at the peak means sensitivity in the wavelength in which the sensitivity becomes the maximum in the same light receiving element, and the sensitivity of 80% means 80% in the comparison with the sensitivity in the wavelength in which the sensitivity becomes the maximum. When the sensitivity of 80% is obtained, a sufficiently clear image is obtained in the comparison with the wavelength in which the sensitivity at the peak is obtained. Under a condition that the sensitivity of less than 80% is obtained, the sensitivity is obtained in the wavelength region from 1000 nm to 1850 nm. Under a condition that the sensitivity of 90% is obtained, the sensitivity is obtained in the wavelength region from 1300 nm to 1780 nm. Thus, in FIG. 2, with the composition ratio of As in the InGaAs of 50 at %, the composition ratio of In of the element B in the InGaAs is 30.8 at % (accuracy of the composition ratio is about ±1.5 at %) and the composition ratio of Ga in the InGaAs is 19.2 at % (accuracy of the composition ratio is about ±2.5 at %).

Additionally, as described later, for a predetermined application for medical treatment, to identify an oil component (lipid) for distinction between a lesion part and a normal part, a threshold of the light receiving element can be set to the element B such that the sensitivity is exhibited from 1000 nm to 1780 nm.

With the composition ratio of arsenide (As) in the composition of InGaAs of 50 at %, the composition ratio (average) of In to Ga of the element C is 31.5:18.5, which is included in the above-described range of theoretical calculation. However, as the actual result of verification with the prototyped element, it was found that the identification of the wavelength region in which the sensitivity of 80% or more in the comparison with the reference value was obtained was difficult. Although the reason is not clear, it is inferred that a degree of lattice mismatch increases in the element C and therefore a function as the light receiving element is deteriorated.

As illustrated in FIG. 3A, while the element A can obtain the sensitivity of almost 100% in the wide wavelength region, the upper limit of the region in which the sensitivity 80% or more of the sensitivity at the peak is obtained is 1640 nm.

Meanwhile, as illustrated in FIG. 3B, the element B may provide sufficient sensitivity for imaging, as described later, although the relative sensitivity of the element B at the wavelength of 1500 nm decreases (100% to about 22%) compared with the light receiving element (the element A in this case) with the composition ratio of In to Ga in the InGaAs in the fourth layer 204 of 28.1:21.9. Additionally, as illustrated in FIG. 3C, the number of saturated electrons and the number of saturated signal electrons of the element B are substantially the same as those of the element A. As a result, in the element B, a value of a S/N ratio is obtained, which does not have large difference compared to that of the element A.

In contrast to this, it was found that the relative sensitivity of the element C significantly decreased (100% to about 0.005%) at the wavelength of 1500 nm, compared with the light receiving element (the element A in this case) with the composition ratio of In to Ga in the InGaAs in the fourth layer 204 of 28.1:21.9, and the element C was found to be difficult to be used as the imaging element. Moreover, as illustrated in FIG. 3C, the element C exhibited the number of saturated electrons and the number of saturated signal electrons lower than those of the element B, and as a result, the S/N ratio also becomes lower than that of the element B.

Here, when the light receiving element 2000 is used for imaging for a predetermined medical treatment application (example: surgery, diagnosis, and the like), the light receiving element 2000 is required to have the sensitivity in the wavelength region at 1700 nm or more. For example, as a method to distinguish between the lesion part and the normal part, which is difficult to be distinguished with visible light in a situation where the lesion part and the normal part are mixed in a part of a human body (example: a tissue and an organ), there is a method using near-infrared light. This is a method that, for example, irradiates an object (a target, such as a tissue of an organism) with near-infrared light and observes the reflected light by an image sensor (light receiving sensor) that has sensitivity to a near-infrared light. Since content rates of water and a lipid are different between the lesion part and the normal part, the lesion part and the normal part can be distinguished using the difference in content rate as an index. While a peak of an absorption coefficient of water is at or around 1450 nm, a peak of an absorption coefficient of a lipid is generally in a wavelength band of 1700 nm or more to 1780 nm or less including at or around 1703 nm, 1730 nm, and 1762 nm (see FIG. 4). Therefore, for example, to graphically identify water content and the lipid in the body and capture an image, the light receiving element is required to have a predetermined sensitivity in the wavelength region at 1700 nm or more.

Accordingly, for use as the medical treatment application as described above, the imaging satisfying the purpose is difficult with the light receiving element (the element A in this case) with the composition ratio (at %) of In to Ga in the InGaAs in the fourth layer 204 of 28.1:21.9. In view of this, this first embodiment employs the element B as the fourth layer 204.

FIG. 5 is a graph illustrating a relationship between irradiance of illuminating light sources required for imaging for the above-described medical treatment application, and wavelengths of the illuminating light for the light receiving elements using the above-described respective elements A, B, and C having the different composition ratios of In to Ga in the fourth layer 204 (the bar charts in FIG. 5 illustrate, for respective wavelengths, the elements A, B, and C in the order from the left). As illustrated in FIG. 5, when the light receiving element 2000 including the element C is attempted to be used for the application for medical treatment, the irradiance of the illuminating light source of the element C exceeds the upper limit value in the safety standard (JIS C 7550). In view of this, regardless of the wavelength region in which the predetermined sensitivity is obtained, the light receiving element using the element C cannot be used for the medical treatment application as described above.

Since the light receiving element using the element A does not have the high sensitivity at the peak of the absorption coefficient of the lipid as described above, the light receiving element A cannot be used for the above-described medical treatment application.

Meanwhile, the light receiving element using the element B has the high sensitivity equivalent to the peak of the absorption coefficient of water at the peak of the absorption coefficient of lipid. Moreover, as illustrated in FIG. 5, the irradiance of the illuminating light required for photographing can be less than the upper limit value of the safety standard.

Although the illustration is omitted, with the composition ratio of As of 50 at % and the composition ratio of in of 31.5 at %, 32 at %, or the like, a state of lattice mismatch progressed, and stability as the light absorbing layer of the light receiving element and pixels was failed to be kept, and thus completing it as the light receiving element was difficult.

Meanwhile, when the composition ratio of As is 50 at % and the composition ratio of In is around 25%, the degree of lattice mismatch is low. However, the upper limit value of the region in which the sensitivity is obtained similarly to the element A fails to reach 1700-plus nm. Thus, there is a problem that an image satisfying the purpose is difficult to be obtained. Accordingly, this embodiment employs the element B (In:Ga=30.8:19.2) at least as the fourth layer 204. Due to this, it is possible to provice a light receiving element and an imaging device that have sufficient sensitivity in the wide wavelength region in the near-infrared region, and have sensitivity in the wavelength region in the further long wavelength area, for example, in excess of 1700 nm. Such element and device may be suitable for certain applications for medical treatment. Accordingly, the element B of the embodiment allows improving quantum efficiency of photoelectric conversion in the wavelength region at 1700 nm or more. As described above, with the composition ratio of As in the InGaAs element of 50 at %, the composition ratio of In in the InGaAs by theoretical calculation is at least 30 at % or more, and the accuracy of In in the composition of InGaAs by analysis is about ±1.5 at %. In this viewpoint, it is judged that the composition ratio of In in the InGaAs is preferably set to be 30 at % or more to less than 31.5 at %. The above-described element B is included in this range.

<Configurations of Imaging System 1 and Imaging Device 3000>

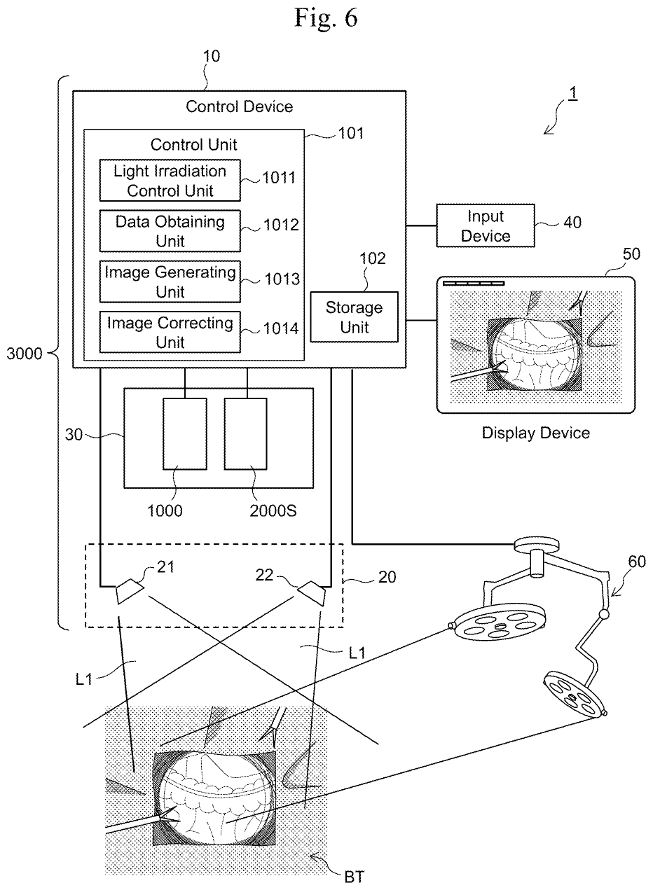

FIG. 6 is a drawing to describe the exemplary configuration of the light receiving element 2000 according to the first embodiment applied to the imaging system 1. The imaging system 1 is used for, for example, medical assistance, such as pathological diagnosis assistance, clinical diagnosis assistance, observation assistance, and surgery assistance (example: an abdominal or laparoscopic surgery system, a surgical robot, and the like). As illustrated in FIG. 6, in this embodiment, a surgery assistance system (imaging system for surgery) will be described as an example of the imaging system 1.

For example, the imaging system 1 includes a control device (control unit) 10, a light source unit 20, an imager (light detection unit) 30, an input device 40, a display device (display unit) 50, and a surgery shadowless lamp 60. The control device 10 controls the whole imaging system 1. The light source unit 20 emits a light with which a tissue BT is irradiated. The imager 30 detects the light emitted from the tissue BT or radiated light (example: reflected light, transmitted light) and captures an image. The input device 40 is used when a user (operator) inputs, for example, various kinds of data and an instruction command to the control device 10. The display device 50 displays, for example, a GUI described later and the image and the like captured by the imager 30. The surgery shadowless lamp 60 is communicatively connected to the control device 10. In this case, the imaging device 3000 includes the control device 10, the light source unit 20, and the imager 30.

The tissue BT is, for example, a laparotomized or exposed organ of, for example, a patient who lies down on a surgical table. The tissue BT can also be referred to as an irradiated body, a sample, and a target.

The control device 10 is configured of, for example, a computer and includes a control unit 101 configured of a processor or the like and a storage unit 102 that stores, for example, various kinds of programs and parameters. The control unit 101 reads, for example, the various kinds of programs and parameters from the storage unit 102, develops the various kinds of programs read into an internal memory (not illustrated), and executes processing of the various kinds of programs in accordance with the instructions input from the input device 40 and information processing sequences identified by the various kinds of programs. The control unit 101 includes, for example, a light irradiation control unit 1011, a data obtaining unit 1012, an image generating unit 1013, and an image correcting unit 1014. The light irradiation control unit 1011 controls the irradiation of the light from the light source unit 20. The data obtaining unit 1012 obtains image data detected (captured) by the imager 30 from the imager 30. The image generating unit 1013 generates an image from the image data obtained from the imager 30. The image correcting unit 1014 corrects the image generated by the image generating unit 1013. The storage unit 102, for example, stores at least programs corresponding to the light irradiation control unit 1011, the data obtaining unit 1012, the image generating unit 1013, and the image correcting unit 1014.

The light source unit 20 includes, for example, a first light source 21 and a second light source 22. The first light source 21 emits (radiates) a visible light with the wavelength from around 380 nm to 750 nm (example: visible light at 550 nm, 650 nm, 700 nm, and the like). The second light source 22 emits (radiates) an infrared light with the wavelength from 800 nm to 3000 nm (example: near-infrared light at 1000 nm, 1300 nm, 1600 nm, 1700 nm, 1730 nm, and the like). While FIG. 6 illustrates an exemplary configuration in which the light source unit 20 includes the two light sources, for example, the light source unit 20 may be configured to disperse a light having a wide-band wavelength range emitted (radiated) from one light source by an optical system, filter the respective dispersed lights by an optical filter disposed on an optical path, and generate a light at a desired wavelength. For example, the light source unit 20 may include a plurality of light sources that emit (radiate) the lights at the wavelengths irradiated to the tissue BT and use the light irradiation from each light source by temporal switching. The operator sets the wavelength of the light emitted (radiated) by the first light source 21 and the wavelength of the light emitted (radiated) by the second light source 22 via the Graphical User Interface (GUI) described later. For example, the control unit 101 reads a value of the wavelength of each light source set with the GUI based on the light irradiation control unit 1011 (program) and transmits a voltage applied to a driving unit (not illustrated) of the light source unit 20 and the wavelength value of the light emitted (radiated) by each light source to this driving unit. Under the control by the control unit 101, this driving unit applies the voltage to the first light source 21 and the second light source 22 to emit (radiate) the light. The control unit 101 controls the irradiances of the illuminating lights (example: visible light, infrared light) from the first light source 21 and the second light source 22 so as to be less than the upper limit value in the safety standard (JIS C 7550). Additionally, for example, the control unit 101 transmits a timing at which the second light source 22 emits (radiates) the light at each wavelength and an emission (radiation) period of the light to the driving unit (not illustrated) of the light source unit 20 and controls the light source unit 20 such that the lights at a plurality of wavelengths are periodically emitted (radiated) from the second light source 22.

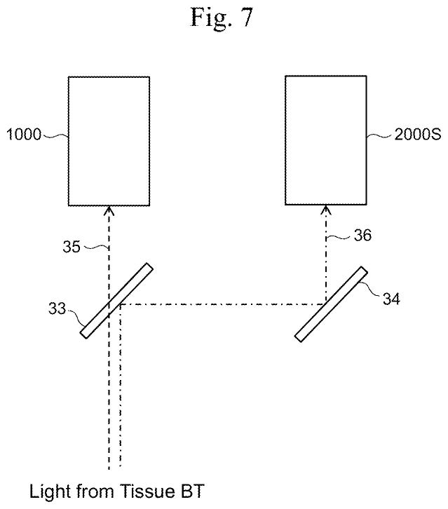

The imager 30 includes a first imaging device 1000 and a second imaging device 200S (imaging element). The first imaging device 1000 irradiates, for example, the tissue BT disposed in a surgical field with a visible light (example: around from 380 nm to 750 nm) to detect a visible light image of the tissue BT. The second imaging device 200S periodically and sequentially irradiates the tissue BT disposed in the surgical field with a light at a second wavelength to a light at a fifth wavelength (here, while the lights at the four kinds of wavelengths are employed, the lights may be lights at two or more kinds or five or more kinds of wavelengths) to detect the lights radiated from the tissue BT. A second imaging device 2000S is an imaging device (imaging element) configured by disposing the plurality of light receiving elements 2000 described above in a matrix on a substrate. Note that the second imaging device 2000S may detect luminance (luminance value) of the light radiated from the tissue BT by irradiation of the light at any of the second wavelength to the fifth wavelength to detect the obtained image (example: an infrared image). The second wavelength to the fifth wavelength are wavelengths longer than the first wavelength, and, for example, four kinds of lights (infrared lights) are selected from the wavelengths from 800 nm to 3000 nm. For example, a silicon (Si) camera can be used as the first imaging device 1000. A camera using the InGaAs for the light receiving element described in FIG. 1 to FIG. 5 can be used for the second imaging device 2000S. As illustrated in FIG. 6, while an optical axis of the first imaging device 1000 and an optical axis of the second imaging device 2000S need not to be the same, as described later using FIG. 7, an optical system that configures the optical axes of both imaging devices to be coaxial may be disposed in the imager 30.

The input device 40 is a device that includes, for example, a keyboard, a computer mouse, a microphone, a touch panel, and the like, and used to input, for example, the instructions and the parameters when the operator (user) causes the control device 10 to execute predetermined processes. Additionally, for example, by simply inserting a semiconductor memory, such as a USB, into an input port (not illustrated) disposed in the control device 10, the control unit 101 in the control device 10 may automatically read data and the instructions (instructions described by a predetermined rule) from the semiconductor memory to execute various kinds of programs.

The display device 50 receives a generated image (for example, a captured image of the sample) generated by the control unit 101 and a corrected image (corrected sample image) obtained by correcting the image (for example, the captured image of the sample) by the control unit 101 from the control device 10 and displays the image, such as the generated image (non-corrected image, the image of the sample) and the corrected image, in a display screen. Note that the display device 50, for example, may combine the generated image (non-corrected image) with the corrected image and output the resultant as an image of the tissue BT (combined sample image). Moreover, the display device 50, for example, can display the generated image (non-corrected image), the corrected image, or the combined sample image in the display screen during surgery. Note that, when generating the combined sample image, for example, while the control unit 101 positions coordinates of the visible light image of the tissue BT with coordinates of the non-corrected image or the corrected image based on, for example, a predetermined positioning mark, the control unit 101 superimposes the visible light image with the non-corrected image or the corrected image to generate the combined sample image.

The surgery shadowless lamp 60 is, for example, a visible light source including a plurality of LED light sources and halogen light sources. The surgery shadowless lamp 60 is considerably bright, for example, in the maximum intensity of 160000 lutes. For example, the control device 10 may control turning on and turning off of the surgery shadowless lamp 60.

<Optical System that Configures Optical Axes of Imaging Devices to be Same>

FIG. 7 is a diagram illustrating a schematic configuration of the optical system employable in the imager 30 in the imaging system 1 (or the imaging device 3000). This optical system is an optical system that configures the optical axis of the first imaging device 1000 and the optical axis of the second imaging device 2000S to be same.

This optical system, for example, can include a dichroic mirror 33 and a mirror 34 as configurations. The dichroic mirror 33 is an optical element (mirror) that has an action of reflecting a light at a specific wavelength (example: infrared light) and causing lights at the other wavelengths (example: visible light) to pass through. For example, by the use of one having a property of causing the visible light to pass through and reflecting the near-infrared light as the dichroic mirror 33, the visible light passes through the dichroic mirror 33 from the tissue BT and enters the first imaging device 1000. Meanwhile, the tissue BT is irradiated with the lights at the four kinds of wavelengths from the second wavelength to the fifth wavelength (for example, the lights selected from the lights at 800 nm to 3000 nm), and the radiated (includes reflection) light is reflected by the dichroic mirror 33 and the mirror 34 and enters the second imaging device 2000S.

The use of the optical system as described above allows configuring the optical axis of the first imaging device 1000 and the optical axis of the second imaging device 2000S to be the same, and this ensures providing an effect that positioning of the images obtained from the two imaging devices is unnecessary.

### Second Embodiment

Next, the second embodiment will be described with reference to the drawings.

The second embodiment employs the light receiving element 2000 similar to that of the first embodiment in an imaging element of an endoscope. Since the light receiving element 2000 has the configuration same as that of the first embodiment, the following omits the overlapping description.

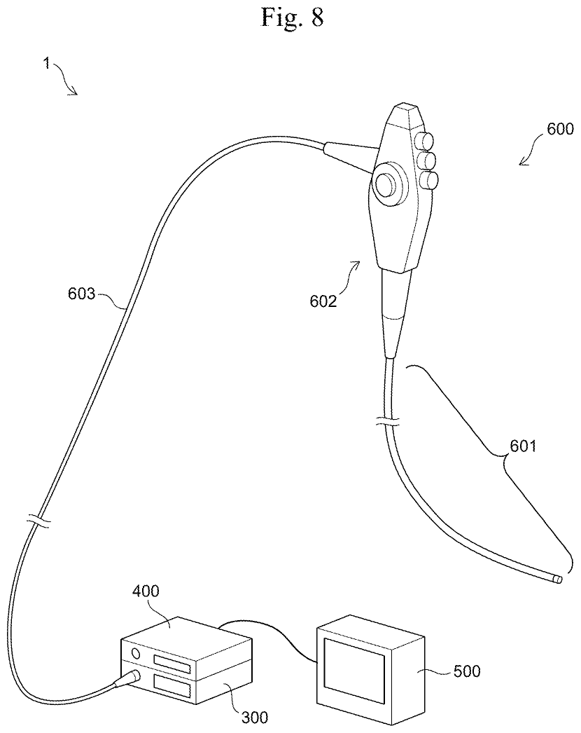

FIG. 8 is an overall configuration diagram of the endoscope system (surgery assistance system) 1 that includes the endoscope.

As illustrated in FIG. 8, the endoscope system 1 of the second embodiment includes an endoscope 600, a light source device 300, a processor 400, and a monitor 500. The endoscope 600 includes imaging means (not illustrated) (example: an imaging element including the plurality of light receiving elements 2000). The light source device 300 supplies an illuminating light. The processor 400 generates a video signal with an electric signal transmitted from the imaging device in the endoscope 600. The monitor 500 is a display device that displays an endoscope image based on this received video signal.

The endoscope 600 of this second embodiment includes a flexible tube 601 inserted into a body of a subject, an operating unit 602 positioned at a base end of this flexible tube 601, and a code 603 extending from one side of this operating unit 602.

The operating unit 602 includes, for example, a curve adjusting knob to adjust a degree of curvature of the flexible tube 601 and various kinds of switches to instruct imaging, water supply, air supply, and the like. The code 603 internally includes, for example, a light guide to guide the illuminating light generated in the light source device 300 and an electric cable to transmit an electric signal from the processor 400.

As described later, the flexible tube 601 internally includes, for example, a wire to adjust a degree of curvature of a distal end portion of the flexible tube 601, a water supply nozzle, a light guide, and an imaging device. The degree of curvature of the distal end portion of the flexible tube 601 is adjusted by the curve adjusting knob disposed in the operating unit 602.

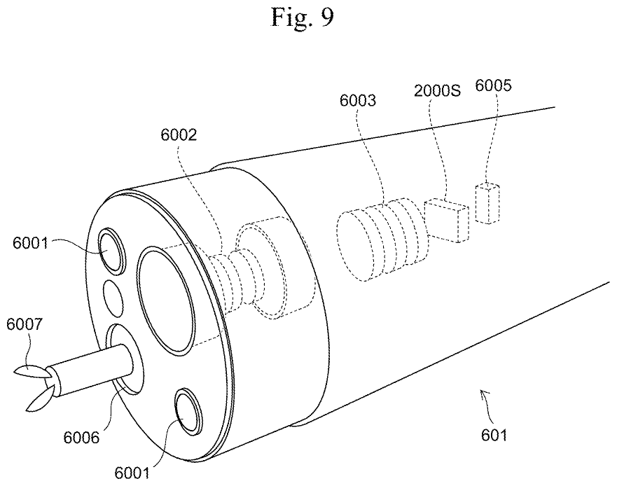

FIG. 9 is a perspective view illustrating an internal configuration of the flexible tube 601. As one example, this flexible tube 601 includes the above-described imaging device 2000S, illumination lenses 6001, an objective lens 6002, an image forming lens 6003, a Peltier element 6005 (cooling element), a channel 6006, and a processing unit 6007 that processes (example: excises, grasps, and the like) the target.

The illumination lenses 6001 are an optical system to guide the illuminating light guided by the light guide (not illustrated), which is disposed inside the flexible tube 601, from the light source device 300 to the outside via the code 603 and the operating unit 602. The light source device 300 in this second embodiment is configured to emit the illuminating light in an infrared region at the wavelength of, for example, from 1000 nm to 2000 nm or from 800 nm to 3000 nm, in addition to the visible light. Note that an illumination optical path to guide the illuminating light of the visible light and an illumination optical path to guide the illuminating light in the infrared region can be disposed separately.

The objective lens 6002 is an optical system to guide a light from a tissue in the body of the subject into the flexible tube 601. The image forming lens 6003 is an optical system to condense the lights from the objective lens 6002 to guide the light to the imaging device 2000S. The Peltier element 6005 is the cooling element to cool the imaging device 2000S. The channel 6006 is a cavity to advance and retreat the processing unit 6007 in the front-rear direction. Note that when the imaging device 2000S having the light receiving element of this embodiment is used, since thermal noise is small and the S/N ratio is high, the cooling element, such as the Peltier element, can be omitted.

The imaging device 2000S may be an imaging element that has a structure same as the second imaging device 2000S of the first embodiment and has sensitivity in the wavelength band from 1000 nm or more to 1850 nm or less as one example. The endoscope system 1 (endoscope 600) with the composition ratio (at %) of In to Ga in the InGaAs in the fourth layer 204 of, for example, 30.8:19.2 allows capturing an image that graphically identifies between the normal part and the lesion part.

### Third Embodiment

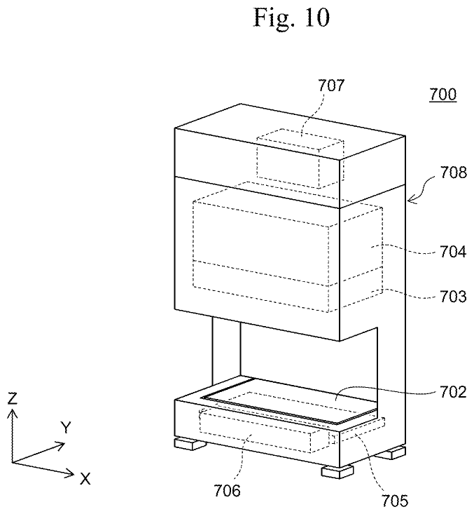

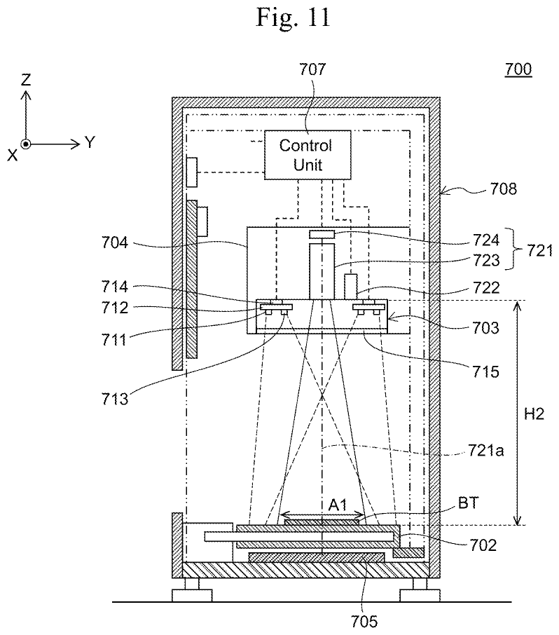

Next, the third embodiment will be described with reference to FIG. 10 and FIG. 11.

The third embodiment employs the light receiving element similar to the light receiving element 2000 of the first embodiment in an imaging device for pathology. Since the light receiving element in an imager 721 described later of the third embodiment is same as that of the light receiving element 2000 of the first embodiment, the following omits the overlapping description.

FIG. 10 in an external view of an imaging device 700 according to the embodiment, and FIG. 11 is a drawing illustrating an internal configuration of the imaging device 700, in an XYZ orthogonal coordinate system in the drawing, for example, the X direction and the Y direction are horizontal directions substantially matching a supporting plane of a sample supporting portion 702, and, for example, the Z direction is a vertical direction perpendicular to the X direction and the Y direction.

The imaging device 700 is, for example, used for medical assistance, such as pathological diagnosis assistance, clinical diagnosis assistance, or observation assistance. As illustrated in FIG. 11, the imaging device 700 includes the sample supporting portion 702, illumination units (illuminators) 703, a detection unit (imaging unit) 704, a calibration reference unit 705, a control unit 707, and a housing portion 708. The sample supporting portion 702 is configured to support a sample including the tissue BT of the organism. The sample supporting portion 702, for example, can be a member having a rectangular plate shape. The sample supporting portion 702, for example, has an upper surface (placement surface) disposed substantially parallel to the horizontal direction and the tissue BT is placeable on this upper surface (placement surface).

The tissue BT may be a tissue of a human or may be a tissue of an organism other than the human (for example, an animal). The tissue BT may be a tissue in a state cut out from the organism or may be a tissue in a state attached to the organism without being cut out. Additionally, the tissue BT may be a tissue (biological tissue) of a living organism (living body), and whether the tissue is a tissue of an organism (cadaver) after death does not matter. The tissue BT may be an object extracted from the organism.

The illumination unit 703 is, for example, disposed above the sample supporting portion 702 and irradiates the tissue BT with the infrared light (near-infrared light). The illumination unit 703 is mounted to, for example, the imaging unit 704. As illustrated in FIG. 11, the illumination unit 703 includes light source units 711, holding members 712, visible light source units 713, and light source moving units 714. The light source unit 711 is configured to emit the infrared light. The holding member 712 is used to hold the light source unit 711. The holding member 712 is, for example, a plate-shaped member and holds the light source unit 711 on its lower surface (a surface opposite to the arrow of the Z direction). Further, the light source moving unit 714 changes an irradiation angle of the infrared light with respect to the tissue BT. The imaging device 700 of this embodiment includes a diffusion member 715. The diffusion member 715 diffuses the infrared light emitted from the light source unit 711. After the diffusion member 715 diffuses the infrared light emitted from the light source unit 711, the tissue BT is irradiated with the infrared light. The illumination unit 703 may be configured to perform shadowless illumination like a shadowless lamp.

In this embodiment, the illumination unit 703 can irradiate the tissue BT with the visible light. The visible light source unit 713 is held to the holding member 712 and emits the visible light. The holding member 712, for example, holds the visible light source unit 713 on its lower surface. The light source moving unit 714 can change the irradiation angle (example: irradiation direction) of the visible light with respect to the tissue BT. After the diffusion member 715 diffuses the visible light emitted from the visible light source unit 713, the tissue BT is irradiated with the visible light.

The diffusion member 715 is disposed so as to cover the emission surface of the illumination units 703. The diffusion member 715 has an opening, which is not illustrated in FIG. 10 or FIG. 11, and an optical path between the imaging unit 704 and the sample supporting portion 702 passes through this opening. Accordingly, the light through the sample supporting portion 702 or the tissue BT passes through this opening and enters the imager (example: the first imager 721, the second imager 722).

A plurality of the illumination units 703 are disposed on a peripheral area of an optical axis (example: the optical axis of the light received by the light receiving element) 721a of the imager (detection unit). The light source unit 711 in each illumination unit 703 includes a plurality of light sources. For example, while each of the plurality of light sources is a light-emitting diode (LED), the light source may include a solid light source, such as a laser diode (LD), or may include a lamp light source, such as a halogen lamp. The plurality of light sources emit infrared lights of mutually different wavelength ranges. The wavelength range of the infrared light emitted from each of the plurality of light sources is selected from, for example, a wavelength band at about 800 nm or more to about 3000 nm or less. While the wavelength ranges of the infrared lights emitted from the plurality of respective light sources are set, for example, so as not to overlap with one another, the wavelength ranges may overlap, and the two or more light sources may emit the infrared lights in the same wavelength range. The number of light sources included in the light source unit 711 in each illumination unit 703 may be one or may be any given number of two or more. While the plurality of light sources are all held by the holding member 712 in each illumination unit 703, the plurality of light sources may be held separately by a plurality of holding members. For example, the plurality of light sources are controlled by the control unit 707 and selectively or collectively emit the infrared lights.

The visible light source unit 713 includes a light source, such as a light-emitting diode (LED). This light source may be a solid light source, such as a laser diode (LD), or may be a lamp light source, such as a halogen lamp. The visible light source unit 713 emits the visible light in the wavelength range at least a part of the wavelength band of, for example, about 380 nm to about 750 nm. The visible light source unit 713 is disposed in, for example, each illumination unit 703. In each illumination unit 703, while the visible light source unit 713 is held to, for example, the holding member 712 same as the plurality of light sources in the light source unit 711, the visible light source unit 713 may be held to a member different from the holding member 712. The number of light sources of the visible light source unit 713 disposed in each illumination unit 703 may be one or two or more. When the visible light source unit 713 includes the plurality of light sources, the wavelength ranges of the visible lights emitted from the plurality of respective light sources may be mutually different between the two or more light sources or may be the same between the two or more light sources.

The light source moving unit 714 changes the irradiation angle of the infrared light with respect to the tissue BT (example: the irradiation direction and the emission direction of the light source unit 711). The irradiation direction of the light source unit 711 is, for example, the direction of the center axis of the infrared light emitted from the light source unit 711. The light source moving unit 714, for example, changes a posture of the holding member 712 to change the irradiation angle of the infrared light with respect to the tissue BT. The irradiation angle of the infrared light from the light source unit 711 is set, for example, such that the positional relation between the light source unit 711 and the first imager 721 is displaced from a relation of regular reflection of the surface of the tissue BT. The irradiation angle of the infrared light from the light source unit 711 may be set such that the positional relation between the light source unit 711 and the first imager 721 is displaced from a relation of regular reflection of the upper surface of the sample supporting portion 702.

The light source moving unit 714, for example, connects the holding member 712 to the imaging unit 704 and moves (for example, turns) the holding member 712 relative to the imaging unit 704. This changes the posture of the holding member 712 and changes the irradiation angle of the infrared light from the light source unit 711. The light source moving unit 714 includes, for example, driving power transmission means, such as a gear, a pulley, and a belt and transmits driving power to move the holding member 712. The light source moving unit 714 may include an actuator, such as an electric motor, that supplies the driving power to move the holding member 712 or needs not to include the actuator. When the light source moving unit 714 includes the actuator, this actuator is controlled by the control unit 707. The control unit 707 may control the light source moving unit 714 to control the irradiation angle of the infrared light. Instead of including the actuator, for example, the operator (user) may manually drive the light source moving unit 714. Additionally, the holding member 712 may be connected (example: supported) to an object different from the imaging unit 704 or needs not to be connected (example: supported) to the imaging unit 704. The light source moving unit 714 may change the irradiation angle of the infrared light for each illumination unit 703 or may collectively change the irradiation angles of the infrared lights by the two or more illumination units 703, by, for example, a link mechanism or the like.

Note that all of the plurality of illumination units 703 have the similar configurations, two or more of the illumination units 703 may have mutually different configurations. For example, one illumination unit 703 may differ from the other illumination unit 703 in at least one of the positional relation of the plurality of light sources to the holding member 712, the number of plurality of light sources, or the wavelength ranges of the infrared lights emitted from the plurality of light sources. The imaging device 700 needs not to include at least a part of the illumination units 703. For example, the illumination unit 703 may be mounted to be exchangeable to the imaging device 700 and may be mounted when the imaging device 700 captures an image. Additionally, at least a part of the illumination units 703 may be, for example, a part of facility (example: interior light) where the imaging device 700 is used.

The imaging unit 704 includes the first imager 721 and a second imager 722. The first imager 721 is, for example, an infrared camera and captures the image of the tissue BT through the reception of the infrared light. The first imager 721 detects a light (example: reflected light, scattered light, transmitted light, reflected scattered light, and the like) radiated from the tissue BT by the irradiation of the infrared light. The first imager 721 includes an imaging optical system (detection optical system) 723 and an imaging element (light receiving element) 724. The imaging optical system 723 includes, for example, an autofocus mechanism (AF mechanism) and forms the image of the tissue BT. The optical axis 721a of the first imager 721 is coaxial with the optical axis of the imaging optical system 723.

The imaging element 724 including the plurality of light receiving elements captures the image formed by the imaging optical system 723. The imaging element 724 includes a two-dimensional image sensor, for example, a CCD image sensor, a CMOS image sensor, and the like. The imaging element 724 has, for example, a structure that has a plurality of two-dimensionally arrayed pixels and disposes an optical detection unit, such as a photodiode, in each pixel. The imaging element 724 may be an imaging element that has the structure same as that of the imaging device 2000S of the first embodiment and has the arrayed light receiving elements having the sensitivity in the wavelength band of 1000 nm or more to 1850 nm or less as one example. The imaging device 700 (imaging element 724) with the composition ratio (at %) of In to Ga in the InGaAs in the fourth layer 204 in the light receiving element of, for example, 30.8:19.2 allows capturing the image that graphically identifies between the lesion part and the normal part.

A detection range A1 (see FIG. 11) of the first imager 721 is, for example, an imaging region on the sample supporting portion 702 where an image can be captured by the first imager 721 and a sight region of the first imager 721 on the sample supporting portion 702. The imaging region of the first imager 721 is, for example, a region optically conjugated with a light-receiving region (region where the optical detection unit is disposed) of the imaging element 724. The sight region of the first imager 721 is, for example, a region optically conjugated with an inside of a field stop of the imaging optical system 723. The first imager 721 generates, for example, data of the captured image, as an imaging result (detection result). The first imager 721 supplies, for example, the data of the captured image to the control unit 707.

The second imager 722 is, for example, a visible camera and captures the image of the tissue BT by reception of the visible light. The second imager 722 detects, for example, the light reflected by and scattered on the surface of the tissue BT among the visible lights from the visible light source units 713. The second imager 722 includes an imaging optical system (not illustrated) and an imaging element (not illustrated). The imaging optical system includes, for example, an autofocus mechanism (AF mechanism) and forms the image of the tissue BT. The imaging element captures the image formed by the imaging optical system. The imaging element includes, for example, a two-dimensional image sensor, such as a CCD image sensor and a CMOS image sensor. The imaging element 724 has, for example, a structure that has a plurality of two-dimensionally arrayed pixels and an arranged optical detection unit, such as a photodiode, in each pixel. The imaging element employs, for example, silicon (Si) for the optical detection unit and has sensitivity in the wavelength range of the visible light emitted from the visible light source unit 713. The second imager 722 generates data of the captured image as an imaging result (detection result). The second imager 722 supplies the data of the captured image to the control unit 707.

The processes and techniques described here are not essentially related to any particular device, and can be implemented by any suitable combination of components. Furthermore, general-purpose, various types of devices can be used in accordance with the method described here. For performing steps of the method described here, it may be beneficial to construct a dedicated device. Further, various inventions can be made by properly combining the plurality of components disclosed in the embodiments. For example, some components may be omitted from all of the components shown in the embodiments. Moreover, components in different embodiments may be suitably combined together.

Other implementation of the present invention will be made apparent for those having ordinary knowledge in the technical field from the examination of the specification and the embodiments of the present invention disclosed herein. The various aspects and/or components of the explained embodiments can be used independently or in any combination.

### REFERENCE SIGNS LIST

1 Imaging system
10 Control device (control unit)
20 Light source unit
21 First light source
22 Second light source
30 Imager (light detection unit)
33 Dichroic mirror
34 Mirror
40 Input device
50 Display device (display unit)
60 Surgery shadowless lamp
101 Control unit
102 Storage unit
201 First layer
202 Second layer
203 Third layer
204 Fourth layer
205 Fifth layer
206 Sixth layer
300 Light source device
400 Processor
500 Monitor
600 Endoscope
601 Flexible tube
602 Operating unit
603 Code
1000 First imaging device
1011 Light irradiation control unit
1012 Data obtaining unit
1013 Image generating unit
1014 Image correcting unit
2000 Light receiving element
2000S Second imaging device
6001 Illumination lens
6002 Objective lens
6003 Image forming lens
6005 Peltier element
6006 Channel
6007 Processing unit

1. A light sensitive element comprising:
a substrate;
a light absorbing layer containing InGaA, the light absorbing layer being disposed on the substrates; and
semiconductor layers containing InAsP, the semiconductor layers being disposed on an upper surface and on a lower surface of the light absorbing layer, respectively, and the semiconductor layers constituting a quantum well structure with the light absorbing layer.

2. The light sensitive element according to claim 1,
wherein when a composition ratio of As in the InGaAs is 50 at %, a composition ratio (at %) of In to Ga in the InGaAs is 30.8:19.2.

3. The light sensitive element according to claim 1,
wherein the light absorbing layer has a sensitivity in a wavelength band of 1000 nm or more to 1850 nm or less.

4. The light sensitive element according to claim 2,
wherein an accuracy of a composition of the In in the InGaAs is about ±1.5 at %.

5. The light sensitive element according to claim 2,
wherein an accuracy of a composition of the Ga in the InGaAs is about ±2.5 at %.

6. The light sensitive element according to claim 1,
wherein layers including at least a layer of the InGaAs and a layer of the InAsP are repeatedly formed.

7. The light sensitive element according to claim 6,
wherein when a composition ratio of the In in the InAsP is 50 at %, a composition ratio (at %) of P and the As in the InAsP disposed on the upper surface of the light absorbing layer is 42:8.

8. The light sensitive element according to claim 1,
wherein a wavelength band of the light absorbing layer in which sensitivity of 80% or more in a comparison with a wavelength in which the sensitivity becomes a maximum is obtained is from 1040 nm or more to 1810 nm or less.

9. The light sensitive element according to claim 1,
wherein the light absorbing layer has a sensitivity in a wavelength band including 1300 nm or more to 1780 nm or less.

10. The light sensitive element according to claim 1,
wherein the light absorbing layer has a sensitivity in a wavelength band including 1700 nm or more to 1780 nm or less.

11. The light receiving element according to claim 1,
wherein the light absorbing layer and the semiconductor layer are each repeatedly formed across a plurality of layers.

12. A light sensitive element comprising:
a substrate;
a light absorbing layer containing InGaA, the light absorbing layer being disposed on the substrate; and
semiconductor layers containing InAsP, the semiconductor layers being disposed on an upper surface and on a lower surface of the light absorbing layer, respectively, and the semiconductor layers constituting a quantum well structure with the light absorbing layer,
wherein the light absorbing layer has a sensitivity in a wavelength band of 1000 nm or more to 1850 nm or less.

13. The light sensitive element according to claim 12,
wherein when a composition ratio of As in the InGaAs is 50 at %, a composition ratio of In in the InGaAs is 30 at % or more to less than 31.5 at %.

14. The light sensitive element according to claim 12,
wherein the light absorbing layer has a sensitivity in a wavelength band including 1300 nm or more to 1780 nm or less.

15. An imaging device comprising:
an imaging element; and
a light source configured to irradiate an object whose image is captured by the imaging element with an illuminating light,
wherein the imaging element includes a plurality of light sensitive elements,
wherein the plurality of light sensitive elements include a light absorbing layer containing InGaA, the light absorbing layer being disposed on the substrate, and semiconductor layers containing InAsP, the semiconductor layers being disposed on an upper surface and on a lower surface of the light absorbing layer, respectively, and the semiconductor layers constituting a quantum well structure with the light absorbing layer.

16. The imaging device according to claim 15,
wherein when a composition ratio of As in the InGaAs is 50 at %, a composition ratio (at %) of In to Ga in the InGaAs is 30.8:19.2.

17. The imaging device according to claim 15,
wherein the light absorbing layer has a sensitivity in a wavelength band of 1000 nm or more to 1850 nm or less.

18. The imaging device according to claim 15,
wherein an accuracy of a composition of the In in the InGaAs is about ±1.5 at %.

19. The imaging device according to claim 18,
wherein an accuracy of a composition of the Ga in the InGaAs is about ±2.5 at %.

20. The imaging device according to claim 19,
wherein when a composition ratio of the In in the InAsP is 50 at %, a composition ratio (at %) of P and the As in the InAsP disposed on the upper surface of the light absorbing layer is 42:8.

21. The imaging device according to any one of claim 15,
wherein a wavelength region of the light absorbing layer in which sensitivity of 80% or more in a comparison with a wavelength in which the sensitivity becomes a maximum is obtained is from 1040 nm or more to 1810 nm or less.

22. The imaging device according to any one of claim 15,
wherein an amount of light irradiation by the light source is less than an upper limit value established by JIS C 7550.

23. An imaging element comprising
the light sensitive element according to claim 1,
wherein the plurality of light sensitive elements are disposed on the substrates.

24. An imaging element comprising the imaging element according to claim 23.
